SEQUENCE LISTING (1 sequence)

SEQ ID NO: 1 - DNA, 53 bp, Artificial
  SEQ ID NO1 is the nucleic acid sequence of the capture base of a hybridization probe.
cttaagacggcgccggcgaccggtttttttccggtcgccgtcgccgccttaag

(19) United States
(12) Patent Application Publication
(10) Pub. No.: US 2005/0074781 A1
(43) Pub. Date: Apr. 7, 2005

(54) Nucleic acid braided J-probes

(75) Inventors: Herbert Peter von Schroeder, Toronto (CA); Larry A. Beard, Mississauga (CA); Xianghai Chen, Toronto (CA)

Correspondence Address: Michael A. Slavin, Esq.;McHale & Slavin, P.A., 2855 PGA Boulevard, Palm Beach Gardens, FL, 33410, US

(73) Assignees: Herbert P. von Schroeder; Larry Beard; Xianghai Chen

(21) Appl. No.: 10/678,726

(22) Filed: Oct. 2, 2003

(51) Int. Cl. C12Q 1/68; C07H 21/04

(52) U.S. Cl. 435/6; 536/24.3

(57) ABSTRACT

This invention relates generally to nucleic acid hybridization and analysis. More specifically, oligonucleotide probes each containing both double and single-stranded regions and one looped end, or arrays of such oligonucleotide probes immobilized on a solid support via the looped end, are provided which are suitable for hybridization and analysis. Based on the molecular structure, specific methods for nucleic acid hybridization, processing, including automated processing, and analysis using the probes or array of probes immobilized on a solid surface, particularly including a reflective surface are also provided.

### FIELD OF THE INVENTION

This invention relates generally to nucleic acid hybridization and analysis. More specifically, a nucleotide probe for hybridization analysis is provided, that comprises an oligonucleotide sequence that consists of a braided J structure attached to a reflective solid surface suitable for processing, detection and analysis of a target nucleotide sequence.

### BACKGROUND OF THE INVENTION

Nucleic acid hybridization, in the forty years since its discovery, has become a powerful tool with implications for biology, medicine and industry. Hybridization assays are based on the very specific base pairing that is found in hybrids of DNA and RNA. Base sequences of analytical interest appearing along a strand of nucleic acid can be detected very specifically and sensitively by observing the formation of hybrids in the presence of a probe nucleic acid known to comprise a base sequence that is complementary with the sequence of interest. Nucleic acid hybridization has been used for a wide variety of purposes including, for example, identification of specific clones from cDNA and genomic libraries, detecting single base pair polymorphisms in DNA, generating mutations by oligonucleotide mutagenesis, amplifying nucleic acids from single cells or viruses, or detecting microbial infections.

Recent advances in nucleic acid hybridization methods have greatly expanded the scope and extent of its potential applications. Of great interest are approaches to miniaturize hybridization reactions by preparing “microarray biochips” (or “DNA chips” or “microarray slides”) containing large numbers of oligonucleotide probes prepared, for example, through VLSIPS™ technology (See U.S. Pat. No. 5,143,854 or 5,561,071). These approaches offer great promise for a wide variety of applications. Microarray biochips are useful for determining nucleic acid by hybridization (see, for example, U.S. Pat. No. 5,741,644), for diagnosis of human immunodeficiency virus (see, for example, U.S. Pat. No. 5,861,242) and for screening potential DNA binding drugs (see, for example, U.S. Pat. No. 5,556,752). Microarrays are commonly used to identify changes in expression of specific genes and gene products.

Microarray technology enables the simultaneous analysis of thousands of sequences of DNA or RNA for genetic and genomic research and for diagnostics. The primary applications of microarrays are for the study of differential gene expression and gene mapping. A single microarray refers to a microscope slide or mesh that has a large number of known probe DNA sequences (also known as oligonucleotides, ESTs, cDNAs) attached thereto. Plural copies of each single target DNA sequence are synthesized or spotted onto a specific location of the microarray. In order to conduct analysis of a specimen, e.g. DNA, one or more test samples containing labeled DNA are allowed to bind to the probe DNAs on the array by a natural process called hybridization. The array is then scanned to determine the presence and/or amount of the labeled test DNA. Identification of the test DNA is based on specific hybridization to the known DNA on the array at its known location.

Use of nucleic acid microarrays generally follow one of two approaches for detecting hybridization to a nucleic acid. Detection can be accomplished if the target nucleic acid is labeled (“direct labeling approach”). Alternatively, detection can be accomplished by a second probe that is detectably labeled and which can hybridize to the nucleic acid of the sample, which is hybridized to the first probe immobilized on the array (“indirect” labeling approach). Either direct or indirect labeling can be utilized with the disclosed invention and processing procedure.

Utilization of microarrays is presently expanding into all aspects of human, animal and plant research at a rapid rate. Arrays are quickly becoming routine tools for high-throughput analysis of gene expression in a wide scope of medical conditions and physiologic systems. The medical applications of microarray technology are not only for molecular diagnostics and gaining insight into mechanisms of disease processes, but also for pharmacological investigations of drug effects and discovering target genes for established and new drugs.

Limitations regarding microarray technology center around concerns of reliability. Repeated experiments and statistical surveys indicate that data deviations can be significant. Commercially available arrays have been scrutinized and array results typically require follow-up confirmation by other molecular biology techniques. Although microarrays are continuing to make a significant impact on research, important technical issues have yet to be resolved.

The overall power and effectiveness of microarray technology thus depends upon two factors (1) the quality of the RNA or DNA to be tested and (2) the quality of the microarray slide. RNA/DNA quality is dependent on the technical expertise of isolation and purification and can be improved by the use of commercially available kits to perform the tasks. The quality of the microarray slide remains dependent on the slide manufacturers. Thus, there exists a longfelt need for microarray slides that maximize slide sensitivity and specificity, and provide highly reproducible results. The instant inventors have achieved this though the use of unique manufacturing processes which provide ideal experimental microarray conditions based on higher and consistent DNA binding capacity, as well as excellent signal-to-noise ratio, thereby improving fluorescent detection and minimizing background signals.

Manufacturing Process Steps:

1. Production of Hairpin capture probe
2. Attachment of capture probe to glass slide
3. Provision of mirror coated glass slide
4. Automation of hybridization and slide washing using sonification.

While analysis of DNA is the illustrative example, the instant technology is equally applicable to the analysis of RNA, protein and other macromolecule samples, and would be useful in combination with Direct RNA labeling and protein and macromolecule arrays.

When referred to throughout this specification, the terms DNA, RNA, protein and macromolecule refer to the particular material or detectable fragments thereof.

Detection of hybridization in a microarray biochip by any labeling technique can be problematic because background hybridization gives rise to a high false-positive assay background. Additionally, when the microarray contains a wide variety of probe sequences for simultaneously detecting a variety of different nucleic acid targets (the reason for miniaturizing hybridization), further cross-reactivity occurs resulting in further false-positive assay background. Accordingly, a need exists for improved hybridization in general and for detecting hybridization on microarray formats in particular. The present invention addresses this and other related needs in the art.

### DESCRIPTION OF THE PRIOR ART

U.S. Pat. No. 5,607,834 (Bagwell) discloses a fluorescent probe for binding to a polynucleotide target and methods using such fluorescent probes that comprises: an oligonucleotide having a segment complementary to the polynucleotide target, the oligonucleotide forming two imperfect hairpins both of which together include the segment except for one nucleotide; and one donor fluorophore and one acceptor fluorophore covalently attached to the oligonucleotide so that only when the imperfect hairpins are formed, the donor fluorophore and the acceptor fluorophore are in close proximity to allow resonance energy transfer therebetween. The fluorescent probes disclosed in Bagwell must contain “imperfect hairpins,” i.e., containing mismatches in the double-stranded stem segment, which opens to bind its target. This change in structure alters the proximity of donor and acceptor fluoroprobes into resulting in a measurable alteration in signal. The teachings of this reference particularly teach away from the instant invention, which uses a perfect hairpin.

U.S. Pat. No. 6,380,377 (Dattagupta) discloses probes which also rely on double stranded regions.

Fujiwara and Oishi, Nucleic Acids Res., 26:5728-5733 (1998) describe a method of covalent attachment of probe DNA to double-stranded target DNA where an imperfect hairpin was used to hybridize to a target DNA. Fujiwara and Oishi describe a DNA hairpin probe and a protocol that is designed for probing double stranded DNA. Their invention differs from that which is instantly disclosed in several ways. They require the use of recA protein and DNA ligase to facilitate DNA binding and hybridization which requires removal of a redundant DNA sequence, see for example FIG. 1 in their manuscript. Furthermore, they do not teach or suggest attachment of the hairpin to a solid surface, but instead merely describe DNA detection based on molecular weight resulting from gel electrophoresis. On the contrary, the instantly disclosed J-probe is designed to bind to fluorescently labeled single stranded target DNA (oligonucleotides, etc.). The instant J-probe attaches to a solid surface to create an array. Based on known sequences of the J-probes, target genes are identified based on their fluorescent signal on specific locations of the microarray.

All of these disclosures differ significantly in design and hybridization kinematics from the present disclosure. In addition, Bagwell does not disclose or teach any immobilized arrays of oligonucleotide probes.

U.S. Pat. No. 5,866,336 (Nazarenko et al.), disclose an oligonucleotide containing a hairpin structure for use as a primer in detecting a target nucleotide sequence.

US Patent Application 2002/0037509 (Arnold et al.) describes attachment of probe to glass.

US Patent Application 2003/0013109 (Ballinger et al.) is directed to the use of hairpins.

US Patent Application 2002/0018996 (Kimura et al.) is directed toward methods of nucleic acid immobilization on a surface. Background describes several methods including chemical bonding (disulfide bond formation) of nucleic acid (DNA) to a surface; or physical adsorption of DNA to nitrocellulose, poly-L-Lysine, or nylon. Methods describe nucleic acid binding at one of its ends to a polymer. Many slide surfaces are described. There is no description of biotin-streptavidin binding, and no description of hairpin or other components of the instant invention.

US Patent Application 2002/0034001 (Faber) describes a reflected light source. No overlap with the instant disclosure of a reflective surface to increase fluorescent signal is evident.

US Patent Application 2002/0094555 (Belotserkovskii et al.) Method to alter DNA sequences, does not overlap the instant disclosure.

US Patent Application 2002/0110819 (Weller et al.) Method describing hybridization between target and probe oligomeric molecules (e.g. DNA) and then separating hybridized duplexes (or unhybridized single strands) via charge-bearing separation medium, does not overlap the instant disclosure.

U.S. Pat. No. 5,374,524 (Miller) describes basic concepts, including biotin-streptavidin.

U.S. Pat. No. 5,688,642 (Chrisey et al.) is directed to a method to attach nucleic acid oligomers (e.g. DNA) to a substrate in specific patterns. This method incorporates irradiation to facilitate DNA-substrate attachment. This method is entirely different from that of the instant invention. Chrisey et al. mention biotin at the non-attached end of the DNA, and it is not a part of their general invention. They mention biotin-streptavidin in general terms relating to separation of DNA sequences, but do not describe in any way the use of biotin-streptavidin for probe-substrate binding. As such, there is no overlap with our inventions.

U.S. Pat. No. 5,770,365 (Lane et al.) describes hairpin type nucleic acid capture moieties which have some similarities to those of the instant invention; however they differ in the optional formation of a “nicked” duplex region, use of a reaction mixture, use of a single biotin molecule and use of a detection probe. These restrictions are not required and are, in fact, undesirable in the instant invention. The instant invention uses a plurality of biotin molecules which increases binding to the solid substrate, thereby increasing the rigor with which the probe/array can be washed, thereby decreasing background signal in improving the overall quality of our product. The ability of the device to withstand rigorous washing techniques (i.e. ultrasonic wash) is a key component to the instant invention.

U.S. Pat. No. 5,994,065 (Van Ness) discloses procedures to prepare solid support surfaces with the intention to reduce background signal, and does not disclose the instant invention.

U.S. Pat. No. 6,114,121 (Fujiwara and Shigemori) discloses a hairpin probe which binds to target nucleic acid. Additional steps and a complex interaction between probe and target results in a specific signal. This patent is similar to the instant disclosure in that specific hairpin probes are used. The differences are that Fujiwara and Shigemori do not utilize the hairpin to attach the probe to a solid surface (their hairpin structure is required for a different purpose). Their probe also must contain a label which is not a requirement of the instant invention. Additionally, their reaction steps, subsequent to hybridization, differ significantly from those of the instant invention.

U.S. Pat. No. 6,380,377 and Application 2003/0082607 (Nanibhushan Dattagupta) describe hairpins that open their double helix section to bind to a target sequence. At least a portion of the capture section is contained in the double stranded portion of the hairpin. The sticky ends of the hairpin are designed to attach to a surface. The design requires that hairpins for different targets are themselves different. In contrast, the loop portion of our hairpin binds to the surface (opposite side to Nanibhushan Dattagupta), all hairpins for all probes are the same, and the capture sequence is single stranded on our probe and readily available for binding.

U.S. Pat. No. 6,403,319 (Lizardi et al.) describes hairpin DNA sequence analogous to the instant invention. Lizardi describes hairpin coupling to detector portion of probe. The focus of the '319 is on the hairpin. The methods of Lizardi et al. involve attachment of the sample nucleotides to the hairpin sequence, then amplifying the sample with the hairpin, altering the conditions to allow the actual hairpin to form, binding the hairpin to a specific probe, and identifying the coupled sample-hairpin-probe complex. The instant invention has significant distinctions, such as use of the hairpin to bind to the solid support surface (Lizardi et al. use a separate probe); use of a unique sequence on the hairpin to detect a sample (Lizardi et al. attach a sample sequence to the hairpin and then detect the complex); and failure to describe a restriction enzyme site.

U.S. Pat. No. 6,413,722 B1 is directed toward polylysine and polyhistidine attachment of probe to substrate and is unrelated to the instant invention.

U.S. Pat. No. 6,426,183 B1 is directed towards OH attachment of probe to glass, and is unrelated to the instant invention.

U.S. Pat. No. 6,432,642 (Livak et al.) is directed toward novel probe and clamp compositions which form a duplex (or triplex) structure used to detect specific nucleic acid sequences. The focus of this patent on detection methods does not in any way overlap with the instant invention. The Livak et al. patent does not address probe-substrate binding.

Similar probes are described in Mergny et al., Nucleic Acids Res., 22:920-928 (1994). Blok and Kramer, Molecular and Cellular Probes, 11: 187-194 (1997) describe an amplification RNA probe containing a molecular switch, i.e., a plurality of hairpin structures. Sriprakash and Hartas, Gene Anal Techn., 6:29-32 (1989) describe a method of generating radioisotope labeled probe with hairpin nucleic acid structure. One common feature of the hairpin structure-containing probes described in the above references is that the nucleotide sequence complementary to a target nucleotide sequence always resides in the single-stranded, not double-stranded, segment of the hairpin structure. While these disclosure are casually related to the present disclosure, significant functional differences in the design of the probe exist, including restriction enzyme site(s), and a looped region that allows biotin-avidin attachment to a solid surface. These fundamental differences allow for unique processing of the probe(s).

### SUMMARY OF THE INVENTION

The braided J-structure includes a single stranded region which is complementary to a target polynucleotide sequence to be detected. The single stranded sequence is attached to a double stranded segment of the braided J structure by a specialized nucleotide region that facilitates combining, and thus changing, the single stranded region. The double stranded region is formed from a linear nucleotide sequence that folds in half by nature of its two sections that consist of perfectly matched nucleotide sequences. At the fold of the double strand, a loop consisting of unpaired nucleotides are bound to a component of biotin-avidin binding system. An array of oligonucleotide probes immobilized via the nucleotide-biotin-avidin binding system on a solid support consisting of a reflective solid surface for hybridization analysis is also provided. Thus, the probes each comprise a nucleotide sequence forming a braided J structure having a nucleotide sequence complementary to a target nucleotide sequence to be detected located within the double stranded segment. These nucleotide sequences may each be classified as nucleotides or polynucleotides. Oligonucleotides are short polymers of nucleotides, generally less than 200 nucleotides, preferably less than 150 nucleotides, more preferably less than 100 nucleotides, more preferably less than 50 nucleotides and most preferably less than 21 nucleotides in length. Polynucleotides are generally considered, in the art, to comprise longer polymers of nucleotides than do oligonucleotides, although there is an art-recognized overlap between the upper limit of oligonucleotide length and the lower limit of polynucleotide length.

With respect to the present invention, “oligonucleotide” generally refers to a nucleic acid, which may comprise a detectable label, that is used as a probe or as a primer; while polynucleotide refers to a nucleic acid containing a target sequence. Consequently, for the purposes of the present invention, the terms “oligonucleotide” and “polynucleotide” shall not be considered limiting with respect to polymer length.

It is an objective of the instant invention to provide an oligonucleotide probe for hybridization analysis, which probe comprises a nucleotide sequence which, under suitable conditions, forms a braided J structure, wherein the double stranded segment of the hairpin structure is formed between two perfectly matched nucleotide sequences, and wherein a single stranded segment is complementary to a target nucleotide sequence to be detected.

It is a further objective of the instant invention to provide an array of oligonucleotide probes immobilized on a solid support for hybridization analysis, which array comprises a solid support suitable for use in nucleic acid hybridization having immobilized thereon a plurality of oligonucleotide probes, each of the probes comprising a nucleotide sequence which, under suitable conditions, forms a braided J structure, wherein at least a portion of the nucleotide single stranded sequence segment is complementary to a target nucleotide sequence to be detected.

It is a still further objective of the instant invention to provide a restriction enzyme site between single and double stranded segments such that specific single stranded regions can be changed thereby allowing different target nucleotide sequence to be detected. Further, the junction between the double and single stranded region is kinematically favorable for the hybridization reaction to occur and thus facilitates high specificity of binding to the target nucleotide sequence to be detected.

It is yet an additional objective of the instant invention to provide a braided J structure which contains a nucleotide loop covalently bound to elements of the biotin-avidin system to allow binding to a solid surface. Binding of the probes to a solid surface by means of the loop at one end of the probe results in alignment of the probes and thus improves binding to the target nucleotide sequence to be detected and reduces cross-hybridization. The biotin-avidin system provides a bond that withstands specific processing, including sonification. The use of sonification removes unbound and loosely bound target sequences and therefore improves detection of target nucleotide sequence to be detected.

It is an additional objective of the present invention to provide a process and a device to automate the process of hybridization and cleaning array slides, thereby providing a time efficient and consistent process.

A further objective of the instant invention is to provide methods for nucleic acid hybridization analysis using the instantly taught probes or array of immobilized probes provided.

Yet a still further objective is to teach methods for processing, including cleaning, the hybridized probes or array of immobilized probes.

An additional objective of the instant invention is to teach a device that automatically controls the hybridization and cleaning processes.

Other objects and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

FIG. 1 depicts the structure of the capture probe base.

### DETAILED DESCRIPTION OF THE INVENTION

Microarray Processes

Hairpin DNA Capture Probe.

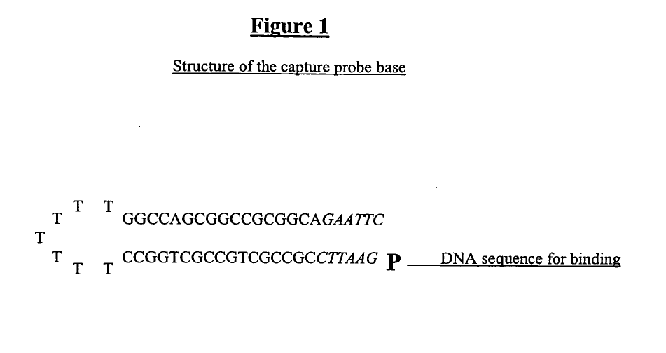

Microarray hybridization is based on the binding of a single strand of test DNA to the single strand of probe DNA. The instantly disclosed probe design consists of a double stranded DNA (referred to as a hairpin or duplex region consisting of a known DNA sequence) to which the standard single stranded probe DNA is attached (FIG. 1). The presence of the hairpin adjacent to the single-stranded probe results in a hybridization to its target DNA with a significant thermodynamic advantage over a similar linear probe. Moreover, the hairpin reduces non-specific DNA binding. Thus, this unique hybridization capture probe significantly enhances the microarray experiments in terms of efficiency, sensitivity and specificity.

DNA microarrays consist of probes (cDNA or oligonucleotide probes) that each have a specific nucleotide sequence that is complimentary to target nucleotides of interest. The latter may be RNA or DNA of genes.

The microarrays that are commercially available differ from each other. These differences include: DNA probes of different lengths (i.e. different numbers of nucleotides), different production methods, different configurations, and different attachments to a solid surface. Target RNA or DNA of the genes of interest are applied to the solid surface; the RNA or DNA binds to the DNA probes of the array by a process of hybridization to form a double helix duplex, and the resulting structure is detected by fluorescence or by radioisotopes to signal the presence of the target sequence.

Array Substrate

The most commonly used substrate for a microarray is a glass slide. However, since DNA does not attach directly to glass, the surface of the substrate must be altered or coated such that the probe does not wash off.

Prior Art Approaches for Attachment have Included:

Ionic attachment—coating a glass slide with amine or lysine allows the positively charged surface to adsorb the negative charge of the phosphodiester backbone of the DNA probe. This results in a two dimensional array;
Covalent attachment—Silane or other aldehyde-containing molecules covalently attach to DNA probes that have amino-modifications. The binding process is termed Schiff's base reaction;
Polymeric attachment—a thin layer of acrylamide polymerize with oligonucleotide probes containing an acrylic acid group (e.g. ACRYDITE from Apogent). A carbon linker is required on the probe. The attachment is simple, but requires several steps. The attachment is thermostable.
Photolithography—oligonucteotides are built on a chemically altered silicone surface (Affymetrix).

In accordance with the present application a process utilizing Biotin-Streptavidin is carried out wherein biotin labeled probes attach to streptavidin coated slides. The biotin-streptavidin attachment mechanism is commonly used in protein chemistry. Its application to DNA/oligonucleotides provides several advantages over other attachment techniques:
(I) Higher consistent binding of DNA. The amount of DNA is consistent throughout a given spot due to the molecular organization of the streptavidin on the slide. The spot-to-spot variability is low with an average spot-to-spot coefficient of variation at 5-7% at all DNA concentrations, substantially lower than any other commercial slides. Consistent binding results in consistent spots and avoids the concerns of variations in capture probe density influencing the interpretation of data;
(ii) Higher DNA binding capacity. Based on binding characteristics, the slides can easily accommodate small volumes (0.05-2 nl/spot) while maintaining spot-to-spot resolution. The unique interaction between DNA and the slide surface efficiently maintains the DNA probe that has been spotted down, which makes a much larger dynamic range of DNA available for hybridization and thus much greater sensitivity;
(iii) Better signal-to-noise ratios in fluorescent detection. Light scatter or background fluorescence will raise the signal-to-noise ratio to a high, often unacceptable level during the scanning step of array processing. The proprietary slide does have a white surface that does scatter incident light during scanning, but this surface does not fluoresce;
(iv) Hybridization—the surface of the slides not only retains greater amounts of DNA probes, but also increases probe availability for hybridization. DNA binding capacity is a critical parameter. The binding between target nucleotide sequences and the probe immobilized on the substrate surface is a bimolecular reaction whose equilibrium depends on the concentration of both reactants;
(v) Slide coating and probe attachment are inexpensive and straight-forward reactions similar to (a) and (b) above, and simpler and less costly than (c) and (d) above. (vi) Thermodynamically and pH resistant biotin-streptavidin binding allows for greater rigor to be used during processing and washing steps, thus reducing background signal and improving target signal.

Slide washing using an ultrasonic bath is an additional component of the present invention.

Probe Design:

cDNA probes (usually 300˜5,000 nucleotides long) or oligonucleotide probes (20˜80 nucleotides long, termed “-mer oligos”) are commonly used for microarrays as simple single-stranded nucleotide chains. Modifications to an end of the chain can be performed for binding to the solid surface, for improving the practicality of adding different nucleotide chains, and for improving the probe-target binding.

The J-probe design of the present application contains a double-stranded hairpin with several features:
(I) A poly-T loop for biotin labeling and solid surface attachment. The loop is located at the end of the probe and thereby allows the probes to align on their spots, and hence create a 3-dimensional array that increases the bioavailability of the probe to the target DNA;
(ii) A double-stranded stem region with phosphorylated ends link to any single or double strand capture sequence. Using DNA ligase, an enzyme that catalyses the formation of phosphodiester bonds, the 5′-phosphate of one double strand oligonucleotide fragment will assemble the 3′-hydroxl terminus on another adjacent single-strand oligonucleotide fragment at 80° C. using a thermostable enzyme DNA-Polymerase I (Klenow fragment). With this technique, a unique capture sequence is added to the J-end. The J portion of the probe is consistent between all probes and therefore standardizes the attachment of the probes to the surface to reduce variability;
(iii) An EcoR I restriction enzyme site on the J probe allows further modification attachment/excision of the unique section of the probe and adds functionality to the probe. It has been demonstrated (Riccelli P V et al. Nucleic Acids Res 2001 Feb. 15;29(4):996-1004) that hairpin probes have higher rates (>2 fold) of hybridization and are more thermodynamically stable compared with linear probes. The physical characteristics of hairpins therefore result in several advantages over standard probes.

Probe Application to a Solid Surface:

Method 1: Spotting involves placing a microscopic droplet of liquid containing the cDNA probe onto the solid surface using a pin. Pins have different tip shapes and the process can be done manually or by robot.

cDNA probes in the applied liquid are dried and thereby immobilized to a solid surface such as glass. This method, “traditionally” called DNA microarray, is widely considered as developed at Stanford University. A variation of this technique involves using ink-jet technology to spray the probe-containing liquid onto the slide. Any of these methods can be employed for the application of our probes to slides.

Method 2: an array of oligonucleotide (20˜80-mer oligos) or peptide nucleic acid probes is synthesized either in situ (directly on the chip) or by conventional synthesis followed by on-chip immobilization. In situ synthesis of the probes is termed photolithographically and was developed by Affymetrix, Inc. The technique involves using light to chemically alter a silicone surface and building nucleotide sequences on the chip. Many companies are manufacturing oligonucleotide based chips using alternative in situ synthesis or depositioning technologies, including inkjet technology in which individual nucleotides are added to a growing probe.

Performance Characteristics:

The performance of the probe depends on the hybridization reaction between probe and target nucleotide. The efficiency of hybridization is dictated by the length of the complimentary sequence, bioavailability of the probe, and attachment of the probe to its support surface. Any alteration of the nucleotide sequence, e.g. by adding fluorescent labels, will also effect hybridization, as will the temperature and general conditions of the environment in which the reaction occurs. Further, the ability to use small samples can reduce error by reducing the amplification (and hence the amplification of error) typically required by standard microarrays. Finally, non-specific binding and background signal will diminish performance of a microarray. Commercially available arrays utilize and manipulate these variables.

Mirror Coating:

The instant invention is unique in utilizing a mirror-coating to increase the fluorescent signal of the array and thereby increases the sensitivity of the array.

Automated Slide Handling Including Ultrasonic Wash:

Existing US patents and patent applications describe the use of ultrasound only for cleaning purposes in array production: e.g. washing pins used to make an arrays, cleaning or altering nebulized spraying during array production, or washing glass substrate in preparation for array production. The instant application differs, in that ultrasonic washes are used to wash the slide to remove unbound and poorly bound target cDNA.

Experiments

Direct comparisons of the instantly disclosed probe have been made to commercially available probes. The J-probe of the instant invention was attached to streptavidin coated slides and compared to conventional commercial cDNA probes (non-J, non-hairpin) attached to non-strep glass slides. The same sample was applied to all slides. Following hybridization, slide processing included ultrasonic washing for intervals up to 1.5 hours.

Results:

J-probe yielded intense signals beyond the measuring capacity of the measuring device. Signal remained within measurable range following washes up to 90 minutes duration. In contrast, these conditions were not conducive to conventional probes which produced only minimal signal that could no longer be detected after washing for 30 minutes or more. These results are summarized in the following table:

TABLE 1

| Run | | | Signal intensity after wash | | | |
|---|---|---|---|---|---|---|
| No. | Probe no. | Probe type | 15 min. wash | 30 min. wash | 60 min. wash | 90 min. wash |
| 1, 2, 8 | 1 to 11 | J on strep | >25,000 | 5000 | 5000 | |
| 3, 4 | 1 to 9 | J on strep | >25,000 | | | 4000 |
| 5, 6 | 1 to 1,200 | Linear (commerc.) | <1000 | not detectable | not detectable | not detectable |

Solid Support Characteristics
A) Substrate: Glass that is mirror-coated on bottom surface to reflect light on its top surface. Streptavidin coating on its top surface for biotin-labelled J-probe attachment.
B) Standard physical parameters and manufacturing are sufficient for the present inventions. The glass slide should be the size of present microscope slides used for the purpose to allow processing in conventional slide holders and scanners used for microarrays. Both surfaces (mirror and streptavidin coating) should be uniform.
   Nucleotide Sequence

Microarray hybridization is based on the binding of a single strand of test DNA to the single strand of probe DNA. The instant probe design consists of a double stranded DNA (referred to as a hairpin or duplex region consisting of a known DNA sequence) to which the standard single stranded probe DNA is attached (FIG. 1). The presence of the hairpin adjacent to the single-stranded probe results in a hybridization to its target DNA with a significant thermodynamic advantage over a similar linear probe. Moreover, the hairpin reduces non-specific DNA binding. Thus, the instant invention's unique hybridization capture probe significantly enhances the microarray experiments in terms of efficiency, sensitivity and specificity.

Specific Features of Hairpin DNA Capture Probe:

The Poly T Loop:

The loop on the left of the hairpin shown in FIG. 1 consists of a sequence of thymine (T) nucleotides. These nucleotides do not bind to themselves. The loop allows for the attachment of other biomolecules (see 2. below) that allow for coupling to a variety of solid support media such as the microarray glass slide.
C. The hairpin. The poly T loop allows the two arms of the DNA to come together. These ends have specific nucleotide sequences that will bind to each other to complete the hairpin. Based in part on the high guanine and cytosine (GC) content, this construct is thermodynamically stable such that it does not unravel during subsequent experimentation.
D. EcoR I restriction enzyme cleavage site (nucleotide sequence shown in italics in FIG. 1). The nucleotide sequence is cleaved by the specific restriction enzyme EcoR I to allow modification of the probe and to allow linkage to any single or double strand capture sequence.
E. Phosphorylated end (bold P in FIG. 1) to allow linkage to any single or double strand capture sequence. Using DNA ligase, an enzyme that catalyses the formation of phosphodiester bonds, the 5′-phosphate of one double strand oligonucleotide fragment will assemble the 3′-hydroxl terminus on another adjacent single-strand oligonucleotide fragment at 80° C. using a thermostable enzyme DNA-Polymerase I (Klenow fragment). The specific DNA probe is thus assembled.

Attachment of Capture Probe to Glass Slide

Standard microarrays consist of individual probes that are haphazardly spotted onto their respective spot on a slide. The instant design allows the hairpin probe to be aligned in its location and provide a consistent density of the probe within its spot. As such, random binding and cross-reactivity are reduced and bioavailability of the probe is increased to maximize hybridization with test DNA. Probe alignment is achieved by specific attachment to the array slide via macromolecules.

Features of Probe Attachment:

a. Biotinylation of poly T loop. During probe manufacturing (sequencing), the three T nucleotides in the center of the T loop (FIG. 1) are bound to the macromolecule Biotin.
b. Slide preparation: glass slides are coated with Streptavidin a nonglycosylated 52,800-dalton protein (concentration 0.2% in filtered coating buffer: 8.0 g NaCl, 0.2 g KCl, 1.44 g Na₂HPO₄, 0.24 g KH₂PO₄ per liter of ddH₂O). Slides are then rinsed and blocked with 2% bovine serum albumin.
c. Probe attachment: Biotinylated hairpin capture probes are spotted or sprayed onto specific locations on the streptavidin coated slide. The probe links to the slide via the biotin-streptavidin attachment. This strong (kd 10¹⁵) attachment has significantly greater stability compared to conventional arrays in which the ligands are passively adsorbed. Through the specific biotin-streptavidin binding, the probes attach to the slide consistently and with a specific alignment to optimize subsequent hybridization as well as scanning.
   Mirror Coated Glass Slide

DNA samples to be tested on the instantly disclosed arrays are prepared to incorporate or attach a fluorescent dye. The labeled DNA then binds to the capture probe and when stimulated with laser light during the scanning process the fluorescent dye allows detection of the DNA sample. Glass slides used in the instantly disclosed arrays have a mirror undercoating that reflects the fluorescent signal emitted from the labeled DNA. The presence of the mirror coating amplifies the signal and allows smaller samples to be tested. This coating increases the sensitivity of the presently disclosed array.

Automation of Hybridization and Slide Washing Using Sonification

We have designed an automated chamber for preparation and hybridization of microarrays. The use of this chamber decreases the handling of the arrays, speeds up the processes, protects the arrays from contamination, provides the optimal environment for the processes to occur, and standardizes the parameters for the processes and subsequent arrays to reduce inter-experimental variability.

Features of the Microarray Processor:

Loading dock for multiple microarray slides.

Automated humidification of microarray slides to prepare slides for sample.

Loading port for DNA sample(s) to be tested.

Automated loading of DNA sample onto microarray slides.

Automated cover applied to microarray slides.

Automated temperature and time control to allow hybridization between sample DNA and microarrays. Removal of cover at completion of step.

Automated removal of unbound DNA off of slide and slide washing using sonification within a specific frequency range. Based on the strong attachment of the probes to the slide via biotin-streptavidin, and based on the strong hybridization of the test DNA to the hairpin probe, our microarrays can withstand an aggressive wash. Sonification results in greater removal on non-specifically bound DNA, unbound DNA and contaminants compared with conventional washing techniques. It is also faster.

Humidity control, time control, temperature control for regulation of all steps.

Centrifuge to spin dry slides.

Analysis of RNA, Protein and Other Macromolecules

Direct RNA Labeling

Techniques described above relate to DNA testing. To test RNA, conventional microarrays require that RNA be converted to DNA. Functionally, our microarrays can be used to test RNA directly, and this is facilitated by (1) the high sensitivity of our arrays and (2) direct labeling of RNA samples based on unique characteristics of RNA. RNA and DNA sequences are each comprised of four nucleotides, however RNA and DNA differ in that RNA contains uracil whereas DNA contains thymine nucleotides. Both uracil and thymine will bind to adenine on a DNA probe.

With specific direct fluorescent labeling of uracil nucleotides on RNA samples, RNA can be detected on microarrays of the present invention.

Protein and Macromolecule Arrays:

The instantly disclosed microarrays can be similarly constructed for the analysis of proteins or other macromolecules. For protein analysis, specific antibodies are used in place of the hairpin probe. The biotinylated antibodies are spotted onto the streptavidin glass slides and allowed to react with labeled proteins. Similarly other macromolecules (e.g. glycoproteins, simple and complex carbohydrates, lipids) can be tested utilizing biotinylated antibodies or probes specific to the macromolecules.

The instantly disclosed microarray technology provides a unique approach to high throughput microarray analysis of DNA, RNA, proteins and other macromolecules. The instantly disclosed arrays bring together sound molecular biology principles that improve individual steps of the array process. By applying this distinctive approach, microarrays having exceptional quality for the growing field of research and diagnostics are provided.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification and drawings.

1. An oligonucleotide probe for hybridization analysis comprising:
a nucleotide sequence forming a braided J structure having both a single and a double stranded segment in which said double stranded segment is formed between two perfectly matched nucleotide sequences, and wherein said single stranded segment is composed of a nucleotide sequence that is complementary to a target polynucleotide sequence to be detected; wherein a region between said single stranded segment and said double stranded segment is linked via nucleotides defining restriction enzyme sites, and wherein said double stranded segment contains an end which defines a nucleotide loop constructed and arranged to facilitate fixation to a solid surface and subsequent processing steps.

2. The probe of claim 1, wherein said oligonucleotide comprises DNA and said target polynucleotide sequence to be detected comprises DNA or a detectable fragment thereof.

3. The probe of claim 1, wherein said oligonucleotide comprises RNA and said target polynucleotide sequence to be detected comprises RNA or a detectable fragment thereof.

4. The probe of claim 1, wherein said oligonucleotide comprises both DNA and RNA and said target polynucleotide sequence to be detected comprises both DNA and RNA and detectable fragments thereof.

5. The probe of claim 1, wherein the double stranded segment is a single stranded nucleotide sequence that has folded onto itself by means of two perfectly matched complementary sequences.

6. The probe of claim 1, wherein said looped end consists of unpaired nucleotides bound to a component of biotin-avidin binding system.

7. An array of oligonucleotide probes immobilized via a biotin-avidin binding system on a solid support for hybridization analysis, comprising:
a solid support suitable for use in nucleic acid hybridization having immobilized thereon a plurality of oligonucleotide probes, each of said probes having a nucleotide sequence that forms a braided J structure having a consistent and thermodynamically stable double stranded segment, wherein the double stranded segment is formed between two complementary nucleotide sequences, and wherein a portion of the probe consists of a single stranded nucleotide sequence located at one end of the double stranded segment which is complementary to a target polynucleotide sequence to be detected, and wherein a region between the single and double stranded segments are linked via nucleotides comprising restriction enzyme sites.

8. The array of claim 7, wherein said plurality of oligonucleotide probes comprise DNA and said target polynucleotide sequence to be detected comprises DNA or a detectable fragment thereof.

9. The array of claim 7, wherein said plurality of oligonucleotide probes comprise RNA and said target polynucleotide sequence to be detected comprises RNA or a detectable fragment thereof.

10. The array of claim 7, wherein said plurality of oligonucleotide probes comprise both DNA and RNA and said target polynucleotide sequence to be detected comprises DNA and RNA and detectable fragments thereof.

11. The array of claim 7, wherein the plurality of probes contain double stranded regions consisting of single stranded nucleotide sequences that have folded onto themselves each by means of two perfectly matched complementary sequences.

12. The array of claim 7, wherein the plurality of probes contain a loop in the folded region that consist of unpaired nucleotides bound to a component of biotin-avidin binding system.

13. The array of claim 7, wherein said plurality of probes attached to a solid surface are processed according to a protocol which includes hybridization and cleaning of the array;
whereby reduction of background and non-specific binding are achieved and said probe becomes capable of withstanding sonification.

14. The array of claim 13 wherein said process protocol includes sonification of said array.

15. The array of claim 13, wherein said process protocol is automated.

16. The array of claim 7 wherein said solid support has a reflective surface or undersurface.

17. The array of claim 16 wherein said reflective surface or undersurface is a mirrored glass.

18. An array of biotinylated antibodies or probes specific to a particular macromolecule immobilized via a biotin-avidin binding system on a solid support for protein or macromolecule analysis, comprising:
a solid streptavidin coated mirrored glass surface having immobilized thereon a plurality of biotinylated antibodies or probes specific for a particular macromolecule.

19. The array of claim 19, further including a labeled protein;
wherein said labeled protein reacts with said biotinylated antibody or probe.

20. The array of claim 18 wherein said macromolecule is selected from the group consisting of a glycoprotein, a simple carbohydrate, a complex carbohydrate and a lipid.
